(19) United States
(12) Patent Application Publication
(10) Pub. No.: US 2019/0275160 A1
(43) Pub. Date: Sep. 12, 2019

(54) BONE-TARGETING THERAPEUTIC CONJUGATE AND METHODS OF MAKING AND USING THE SAME

(71) Applicant: The Regents of the University of California, Oakland, CA (US)

(72) Inventors: B. Chia Soo, Beverly Hills, CA (US); Ting Kang, Beverly Hills, CA (US); Benjamin M. Wu, San Marino, CA (US)

(21) Appl. No.: 16/084,525

(22) PCT Filed: Mar. 14, 2017
(86) PCT No.: PCT/US17/22324

§ 371 Date: Sep. 12, 2018

Related U.S. Application Data
(60) Provisional application No. 62/308,757, filed on Mar. 15, 2016.

(51) Int. Cl. A61K 47/54; A61K 47/64; A61K 38/17; A61K 38/18; A61P 19/10; A61K 47/60

(52) U.S. Cl. CPC A61K 47/548; A61K 47/64; A61K 47/60; A61K 38/1875; A61P 19/10; A61K 38/179

(57) ABSTRACT

The present invention discloses a bone-targeting therapeutic conjugate comprising a formula of TG-M-D (I) or M-D-TG (II) and methods of making and using the same. The present invention also discloses a composition comprising the conjugate and methods of making and using the composition.

### CROSS-REFERENCE TO RELATED APPLICATIONS

This application claims the benefit of U.S. Provisional Application No. 62,308,757, filed on Mar. 15, 2016, the teaching of which is incorporated by reference in its entirety.

### STATEMENT OF GOVERNMENTAL SUPPORT

This invention was made with Government support under AR066782, awarded by the National Institutes of Health. The Government has certain rights in the invention.

### FIELD OF THE INVENTION

The present invention generally relates a bone-targeting therapeutic conjugate and methods of making and using the same and compositions thereof and methods of making and using the composition.

### BACKGROUND OF THE INVENTION

With an aging population, the biomedical burden of osteoporosis is significantly escalating, with no novel therapeutic to address systemic bone loss. Substantial efforts are drawn to developing new strategies to address systemic bone conditions with limited success. For example, NELL-1 is an osteoinductive factor recently discovered to induce bone formation and reverse osteoporotic bone loss when administered intravenously. However, unmodified NELL-1 requires an impractical 48-hour injection frequency and thus limits NELL-1's translation into a clinical setting.

Therefore, there is a continuing need for strategies and agents for treating or ameliorating osteoporosis. The embodiments below address the above described problems and needs.

### SUMMARY OF THE INVENTION

In one aspect of the present invention, it is provided a bone-targeting therapeutic conjugate, comprising a formula of TG-M-D (I) or M-D-TG (II), wherein:

TG is a bone targeting molecule,

D is a therapeutic drug for a bone related condition, and

M is an optional chemical group that modifies the therapeutic drug.

In some embodiments of the invention conjugate, optionally in combination with any one or all of the various embodiments disclosed herein, the bone targeting molecule is a bisphosphate or DSS peptide.

In some embodiments of the invention conjugate, optionally in combination with any one or all of the various embodiments disclosed herein, the bone targeting molecule is a bisphosphonate comprising a chemical formula:

(OX)₂(O)P—(CR₁R₂)ₙ—P(O)(OM)₂, wherein:
each X is independently H, or a cation,
each R₁ is independently a hydroxyl, amino, thiol, amide, or carboxyl group,
each R₂ is a C1-C20 group comprising optional hetero atom(s), and
n is an integer ranging from 1-10.

In some embodiments of the invention conjugate, optionally in combination with any one or all of the various embodiments disclosed herein, the bisphosphonate is one of:

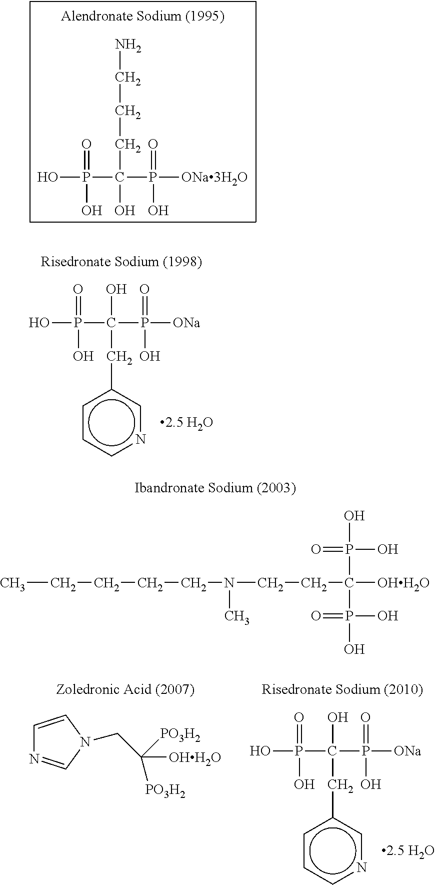

In some embodiments of the invention conjugate, optionally in combination with any one or all of the various embodiments disclosed herein, the therapeutic drug is a small molecule drug.

In some embodiments of the invention conjugate, optionally in combination with any one or all of the various embodiments disclosed herein, the therapeutic drug is a proteineous drug.

In some embodiments of the invention conjugate, optionally in combination with any one or all of the various embodiments disclosed herein, the proteineous drug is NELL-1 protein or a BMP protein.

In some embodiments of the invention conjugate, optionally in combination with any one or all of the various embodiments disclosed herein, the optional chemical group comprises an alkyl group, an aryl group, an acyl group, a leaving group, a polymer, a peptide, or a combination thereof.

In some embodiments of the invention conjugate, optionally in combination with any one or all of the various embodiments disclosed herein, the optional chemical group is poly(ethylene glycol) (PEG), poly(ethylene oxide) (PEO), poly(propylene glycol) (PPG), or poly(propylene oxide) (PPO).

In some embodiments of the invention conjugate, optionally in combination with any one or all of the various embodiments disclosed herein, the optional chemical group is selected from heparin sulfate, glycopolymers, zwitterionic polymers, hyperbranced polymers, polymers containing unnatural amino acids, linkers to lysine or cysteine, or peptide sequences that modify drug or conjugate interactions with ECM, target cells, immune cells, and hepatocytes.

In some embodiments of the invention conjugate, optionally in combination with any one or all of the various embodiments disclosed herein, the optional chemical group comprises a responsive linker that degrades on demand to external stimuli.

In some embodiments of the invention conjugate, optionally in combination with any one or all of the various embodiments disclosed herein, the optional chemical group provides a linking reaction that is azide-alkyne, azide-BMCO, or Tetrazine-TCO type reactions.

In some embodiments of the invention conjugate, optionally in combination with any one or all of the various embodiments disclosed herein, the optional chemical group comprises a linker to lysine or cysteine.

In some embodiments of the invention conjugate, optionally in combination with any one or all of the various embodiments disclosed herein, the optional chemical group comprises a natural enzyme.

In another aspect of the present invention, it is provided a composition, comprising a bone-targeting therapeutic conjugate according to any of the various embodiments disclosed herein.

In some embodiments of the invention composition, optionally in combination with any one or all of the various embodiments disclosed herein, the composition further comprises a pharmaceutically acceptable carrier.

In some embodiments of the invention composition, optionally in combination with any one or all of the various embodiments disclosed herein, the composition is a formulation for systemic or local delivery.

In another aspect of the present invention, it is provided a method of preparing a bone-targeting therapeutic conjugate of a formula of TG-M-D (I) or M-D-TG (II), comprising:

a) providing a therapeutic drug for a bone related condition,

b) providing a bone-targeting molecule,

c) providing an optional compound comprising a chemical group that modifies the therapeutic drug,

d) forming the conjugate,

wherein the conjugate is according to any one of any of the various embodiments disclosed herein.

In another aspect of the present invention, it is provided a method of forming a composition, comprising

providing an amount of a bone-targeting therapeutic conjugate of a formula of TG-M-D (I) or M-D-TG (II), and

forming the composition,

wherein the conjugate is according to any one of any of the various embodiments disclosed herein.

In some embodiments of the invention method, optionally in combination with any one or all of the various embodiments disclosed herein, the composition further comprises a pharmaceutically acceptable carrier.

In another aspect of the present invention, it is provided a method of treating or ameliorating a condition in a subject, comprising administering to the subject a bone-targeting therapeutic conjugate of a formula of TG-M-D (I) or M-D-TG (II),

wherein the conjugate is according to any one of any of the various embodiments disclosed herein.

In some embodiments of the invention method, optionally in combination with any one or all of the various embodiments disclosed herein, the conjugate is included in a composition that comprises the conjugate and a pharmaceutically acceptable carrier.

In some embodiments of the invention method, optionally in combination with any one or all of the various embodiments disclosed herein, the bone related condition is osteoporosis.

In some embodiments of the invention method, optionally in combination with any one or all of the various embodiments disclosed herein, the bone related condition is bone fracture or intervertebral disc disease or injury.

In some embodiments of the invention method, optionally in combination with any one or all of the various embodiments disclosed herein, administering comprises local or systemic administration.

In some embodiments of the invention method, optionally in combination with any one or all of the various embodiments disclosed herein, the subject is a human being.

### BRIEF DESCRIPTION OF THE DRAWINGS

FIG. 1 shows the results of HA binding test which shows the affinity of embodiment conjugates of invention.

FIG. 2 shows that difference between DS S-NELL/DS S-PEG-NELL is smaller than BP-NELL/BP-PEG-NELL

FIG. 3 shows the results of studies on the thermal stability of different targeting NELL-1 of invention.

FIG. 4 shows the results of studies on the biodistribution at 48 h of embodiment conjugates of invention against control.

FIG. 5 shows the results of studies on the biodistribution of embodiment conjugates of invention against control.

### DETAILED DESCRIPTION OF THE INVENTION

### Definitions

The term “therapeutically effective amount”, as used herein, is an amount of an agent that is sufficient to produce a statistically significant, measurable change of a condition in repaired tissue using the agent disclosed herein as compared with the condition in the repaired tissue without using the agent. Such effective amounts can be gauged in clinical trials as well as animal studies. Such a statistically significant, measurable, and positive change of a condition in repaired tissue using the agent disclosed herein as compared with the condition in the repaired tissue without using the agent is referred to as being an “improved condition”.

As used herein, the term “significantly” or “significant” shall mean statistically significant.

Whenever referred to, the term “chemical group” refers to molecular or polymeric chemical or biochemical compound, which can be natural or synthetic. The chemical compound can include any of the groups disclosed herein above or below.

Whenever referred to, the term “alkyl” whenever used refers to a monovalent alkane (hydrocarbon) derived radical containing from 1 to 10 carbon atoms unless otherwise defined. It may be straight, branched or cyclic. Preferred alkyl groups include methyl, ethyl, propyl, isopropyl, butyl, t-butyl, cyclopentyl and cyclohexyl. When substituted, alkyl groups may be substituted with up to four substituent groups, selected from Rd and Ri, as defined, at any available point of attachment When the alkyl group is said to be substituted with an alkyl group, this is used interchangeably with “branched alkyl group”.

Whenever referred to, cycloalkyl is a specie of alkyl containing from 3 to 15 carbon atoms, without alternating or resonating double bonds between carbon atoms. It may contain from 1 to 4 rings which are fused.

Whenever referred to, the term “alkenyl” refers to a hydrocarbon radical straight, branched or cyclic containing from 2 to 10 carbon atoms and at least one carbon to carbon double bond. Preferred alkenyl groups include ethenyl, propenyl, butenyl and cyclohexenyl.

Whenever referred to, the term “alkynyl” refers to a hydrocarbon radical straight or branched, containing from 2 to 10 carbon atoms and at least one carbon to carbon triple bond. Preferred alkynyl groups include ethynyl, propynyl and butynyl.

Whenever referred to, aryl refers to aromatic rings e.g., phenyl, substituted phenyl and the like, as well as rings which are fused, e.g., naphthyl, phenanthrenyl and the like. An aryl group thus contains at least one ring having at least 6 atoms, with up to five such rings being present, containing up to 22 atoms therein, with alternating (resonating) double bonds between adjacent carbon atoms or suitable heteroatoms. The preferred aryl groups are phenyl, naphthyl and phenanthrenyl. Aryl groups may likewise be substituted as defined. Preferred substituted aryls include phenyl and napbthyl.

Whenever referred to, the term “heteroaryl” refers to a monocyclic aromatic group having 5 or 6 ring atoms, or a bicyclic aromatic group having 8 to 10 atoms, containing at least one heteroatom, O, S or N, in which a carbon or nitrogen atom is the point of attachment, and in which one or two additional carbon atoms is optionally replaced by a heteroatom selected from O or S, and in which from 1 to 3 additional carbon atoms are optionally replaced by nitrogen heteroatoms, said heteroaryl group being optionally substituted as described herein. Examples of this type are pyrrole, pyridine, oxazole, thiazole and oxazine. Additional nitrogen atoms may be present together with the first nitrogen and oxygen or sulfur, giving, e.g., thiadiazole.

Whenever referred to, teteroarylium refers to heteroaryl groups bearing a quaternary nitrogen atom and thus a positive charge.

When a charge is shown on a particular nitrogen atom in a ring which contains one or more additional nitrogen atoms, it is understood that the charge may reside on a different nitrogen atom in the ring by virtue of charge resonance that occurs.

Whenever referred to, the term “heterocycloalkyl” refers to a cycloalkyl group (nonaromatic) in which one of the carbon atoms in the ring is replaced by a heteroatom selected from O, S or N, and in which up to three additional carbon atoms may be replaced by hetero atoms.

Whenever referred to, the terms “quaternary nitrogen” and “positive charge” refer to tetravalent, positively charged nitrogen atoms including, e.g., the positively charged nitrogen in a tetraalkylammonium group (e.g. tetramethylammonium), heteroarylium, (e.g., N-methyl-pyridinium), basic nitrogens which are protonated at physiological pH, and the like. Cationic groups thus encompass positively charged nitrogen-containing groups, as well as basic nitrogens which are protonated at physiologic pH.

Whenever referred to, the term “heteroatom” means O, S or N, selected on an independent basis.

Whenever referred to, halogen and “halo” refer to bromine, chlorine, fluorine and iodine. Whenever referred to, alkoxy refers to C1-C4 alkyl-O—, with the alkyl group optionally substituted as described herein.

Whenever referred to, guanidinyl refers to the group: H2NC(NH)NH—.

Whenever referred to, carbamimidoyl refers to the group: H2NC(NH)—.

Whenever referred to, ureido refers to the group: H2NC(O)NH—.

When a group is termed “substituted”, unless otherwise indicated, this means that the group contains from 1 to 4 substituents thereon. With respect to R, Ra, Rb and Rc, the substituents available on alkyl groups are selected from the values of Rd. Many of the variable groups are optionally substituted with up to four Ri groups. With respect to Re, Rf and Rg, when these variables represent substituted alkyl, the substituents available thereon are selected from the values of Ri.

When a functional group is termed “protected”, this means that the group is in modified form to preclude undesired side reactions at the protected site. Suitable protecting groups for the compounds of the present invention will be recognized from the present application taking into account the level of skill in the art, and with reference to standard textbooks, such as Greene, T. W. et al. Protective Groups in Organic Synthesis Wiley, New York (1991). Examples of suitable protecting groups are contained throughout the specification.

Whenever present, in some of the embodiments of the present invention, M can be used to denote a readily removable carboxyl protecting group, and/or P can be used to denote a hydroxyl which is protected by a hydroxyl-protecting group. Such conventional protecting groups consist of known groups which are used to protectively block the hydroxyl or carboxyl group during the synthesis procedures described herein. These conventional blocking groups are readily removable, i.e., they can be removed, if desired, by procedures which will not cause cleavage or other disruption of the remaining portions of the molecule. Such procedures include chemical and enzymatic hydrolysis, treatment with chemical reducing or oxidizing agents under mild conditions, treatment with a transition metal catalyst and a nucleophile and catalytic hydrogenation.

Examples of carboxyl protecting groups include allyl, benzhydryl, 2-naphthylmethyl, benzyl, silyl such as t-butyldimethylsilyl (TBDMS), phenacyl, p-methoxybenzyl, o-nitrobenzyl, p-methoxyphenyl, p-nitrobenzyl, 4-pyridylmethyl and t-butyl.

Examples of suitable C-6 hydroxyethyl protecting groups include triethylsilyl, t-butyldimethylsilyl, o-nitrobenzyl-oxycarbonyl, p-nitrobenzyloxycarbonyl, benzyloxycarbonyl, allyloxycarbonyl, t-butyloxycarbonyl, 2,2,2-trichloroethyloxy-carbonyl and the like.

Whenever present, with respect to —CO2M, which is attached to the carbapenem nucleus at position 3, this represents a carboxylic acid group (M represents H), a carboxylate anion (M represents a negative charge), a pharmaceutically acceptable ester (M represents an ester forming group) or a carboxylic acid protected by a protecting group (M represents a carboxyl protecting group).

Whenever present, the pharmaceutically acceptable salts referred to above may take the form —COOM, where M is a negative charge, which is balanced by a counter ion, e.g., an alkali metal cation such as sodium or potassium. Other pharmaceutically acceptable counter ions may be calcium, magnesium, zinc, ammonium, or alkylammonium cations such as tetramethylammonium, tetrabutylammonium, choline, triethylhydroammonium, meglumine, or triethanolhydroammonium, etc.

Whenever present, the pharmaceutically acceptable salts referred to above also include acid addition salts. Thus, the Formula I compounds can be used in the form of salts derived from inorganic or organic acids. Included among such salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, pamoate, pectinate, persulfate, 3-phenylpropionate, picrate, pivalate, propionate, succinate, tartrate, thiocyanate, tosylate and undecanoate.

Whenever referred to, the pharmaceutically acceptable esters are such as would be readily apparent to a medicinal chemist, and include, for example, those described in detail in U.S. Pat. No. 4,309,438. Included within such pharmaceutically acceptable esters are those which are hydrolyzed under physiological conditions, such as pivaloyloxymethyl, acetoxymethyl, phthalidyl, indanyl and methoxymethyl, and others described in detail in U.S. Pat. No. 4,479,947. These are also referred to as “biolabile esters”.

Whenever referred to, biolabile esters are biologically hydrolizable, and may be suitable for oral administration, due to good absorption through the stomach or intestinal mucosa, resistance to gastric acid degradation and other factors. Examples of biolabile esters include compounds in which M represents an alkoxyalkyl, alkylcarbonyloxyalkyl, alkoxycarbonyloxyalkyl, cycloalkoxyalkyl, alkenyloxyalkyl, aryloxyalkyl, alkoxyaryl, alkylthioalkyl, cycloalkylthioalkyl, alkenylthioalkyl, arylthioalkyl or alkylthioaryl group. These groups can be substituted in the alkyl or aryl portions thereof with acyl or halo groups. The following M species are examples of biolabile ester forming moieties.: acetoxymethyl, 1-acetoxyethyl, 1-acetoxypropyl, pivaloyloxymethyl, 1-isopropyloxycarbonyloxyethyl, 1-cyclohexyloxycarbonyloxyethyl, phthalidyl and (2-oxo-5-methyl-1,3-dioxolen-4-yl) methyl.

Whenever present, L- can be present or absent as necessary to maintain the appropriate charge balance. When present, L- represents a pharmaceutically acceptable counter ion. Most anions derived from inorganic or organic acids are suitable. Representative examples of such counter ions are the following: acetate, adipate, aminosalicylate, anhydromethylenecitrate, ascorbate, aspartate, benzoate, benzenesulfonate, bromide, citrate, camphorate, camphorsulfonate, chloride, estolate, ethanesulfonate, fumarate, glucoheptanoate, gluconate, glutamate, lactobionate, malate, maleate, mandelate, methanesulfonate, pantothenate, pectinate, phosphate/diphosphate, polygalacturonate, propionate, salicylate, stearate, succinate, sulfate, tartrate and tosylate. Other suitable anionic species will be apparent to the ordinarily skilled chemist.

Likewise, when L- represents a specie with more than one negative charge, such as malonate, tartrate or ethylenediamine-tetraacetate (EDTA), an appropriate number of carbapenem molecules can be found in association therewith to maintain the overall charge balance and neutrality.

As used herein, the term “optional” shall mean having the choice to add or not to add a technical element or feature to an embodiment of invention. As such, the term “optional” can also be construed to mean “with” or “without” a technical element or feature in an embodiment of invention.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

As used herein, the term “desirable property” refers to any attributes of a biologies that is significant with respect to the biologies' action as a therapeutics or biologically active agent. Such desirable properties include, for example, blood circulation life, shelf-life, hydrophobicity or hydrophilicity, biological activity, bioavailability, cytotoxicity, non-immunogenicity, or conformational properties, etc.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

Bone-Targeting Therapeutic Conjugate

In one aspect of the present invention, it is provided a bone-targeting therapeutic conjugate, comprising a formula of TG-M-D (I) or M-D-TG (II), wherein:

TG is a bone targeting molecule,

D is a therapeutic drug for a bone related condition, and

M is an optional chemical group that modifies the therapeutic drug.

In some embodiments of the invention conjugate, optionally in combination with any one or all of the various embodiments disclosed herein, the bone targeting molecule is a bisphosphate or DSS peptide.

In some embodiments of the invention conjugate, optionally in combination with any one or all of the various embodiments disclosed herein, the bone targeting molecule is a bisphosphonate comprising a chemical formula:

(OX)₂(O)P—(CR₁R₂)ₙ—P(O)(OM)₂, wherein:

each X is independently H, or a cation,

each R₁ is independently a hydroxyl, amino, thiol, amide, or carboxyl group,

each R₂ is a C1-C20 group comprising optional hetero atom(s), and

n is an integer ranging from 1-10, e.g., 1, 2, 3, 4, 5, 6, 7, 8, 9 or 10.

In some embodiments of the invention conjugate, optionally in combination with any one or all of the various embodiments disclosed herein, the bisphosphonate is one of:

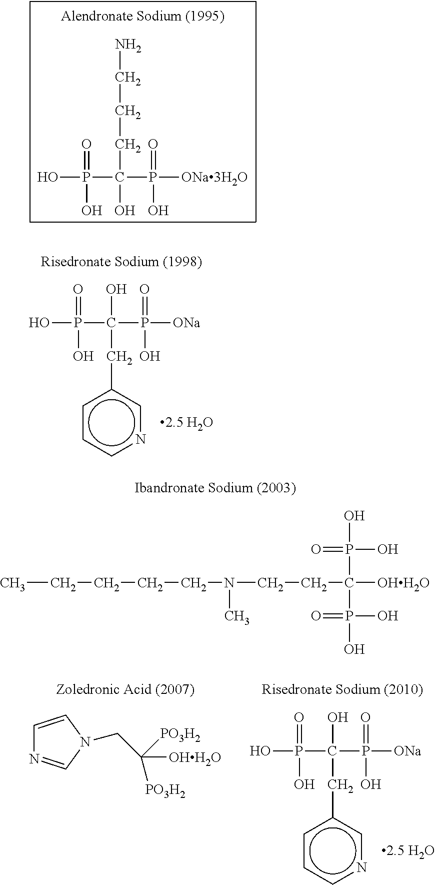

In some embodiments of the invention conjugate, optionally in combination with any one or all of the various embodiments disclosed herein, the therapeutic drug is a small molecule drug.

In some embodiments of the invention conjugate, optionally in combination with any one or all of the various embodiments disclosed herein, the therapeutic drug is a proteineous drug.

In some embodiments of the invention conjugate, optionally in combination with any one or all of the various embodiments disclosed herein, the proteineous drug is NELL-1 protein or a BMP protein.

In some embodiments of the invention conjugate, optionally in combination with any one or all of the various embodiments disclosed herein, the optional chemical group comprises an alkyl group, an aryl group, an acyl group, a leaving group, a polymer, a peptide, or a combination thereof.

In some embodiments of the invention conjugate, optionally in combination with any one or all of the various embodiments disclosed herein, the optional chemical group is poly(ethylene glycol) (PEG), poly(ethylene oxide) (PEO), poly(propylene glycol) (PPG), or poly(propylene oxide) (PPO).

In some embodiments of the invention conjugate, optionally in combination with any one or all of the various embodiments disclosed herein, the optional chemical group is selected from heparin sulfate, glycopolymers, zwitterionic polymers, hyperbranced polymers, polymers containing unnatural amino acids, linkers to lysine or cysteine, or peptide sequences that modify drug or conjugateinteractions with ECM, target cells, immune cells, and hepatocytes.

In some embodiments of the invention conjugate, optionally in combination with any one or all of the various embodiments disclosed herein, the optional chemical group comprises a responsive linker that degrades on demand to external stimuli.

In some embodiments of the invention conjugate, optionally in combination with any one or all of the various embodiments disclosed herein, the optional chemical group provides a linking reaction that is azide-alkyne, azide-BMCO, or Tetrazine-TCO type reactions.

In some embodiments of the invention conjugate, optionally in combination with any one or all of the various embodiments disclosed herein, the optional chemical group comprises a linker to lysine or cysteine.

In some embodiments of the invention conjugate, optionally in combination with any one or all of the various embodiments disclosed herein, the optional chemical group comprises a natural enzyme.

In some embodiments of the invention conjugate, optionally in combination with any or all of the various embodiments disclosed herein, the at least one chemical group imparts at least one desirable property to the therapeutic drug such that the conjugate is significantly improved in the at least one desirable property relative to a naked therapeutic drug (e.g., NELL-1 protein) without chemical modification.

In some embodiments of the invention conjugate, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

As used herein, the term “significantly” in connection with the phrase “significantly improved” shall mean “statistically significant” and, in certain embodiments, can mean an improvement of 10% or above, 20% or above, 30% or above, 40% or above, 50% or above, 60% or above, 70% or above, 80% or above, 90% or above, 100% or above, 200% or above, 300% or above, 400% or above, 500% or above, 600% or above, 700% or above, 800% or above, 900% or above, or 1000% or above in a desirable property of a modified therapeutic drug over that of the unmodified or naked therapeutic drug.

### NELL-1 Protein

NEL-like molecule-1 (NELL-1) protein is widely studied in bone regeneration as an osteogenic growth factor with higher specificity to osteoblast cells compared to the growth factors currently used such as BMP-2. NELL-1 is a secreted homotrimer protein with molecular weight up to 400 KDa. The subunit of NELL-1 contains 810 amino acids and a molecular weight of about 90 KDa before N-glycosylation and oligomerisation. Previous studies suggested that NELL-1 can specifically modulate the osteochondral lineage and induce bone formation in various kinds of animal models from rodents to sheep. Recently, Kwak et al., have demonstrated that the locally intramedullary application of NELL-1 in the femurs of ovariectomy (OVX)-induced osteoporotic female rats could enhance rat bone quality and prevent osteoporosis. In vivo studies further indicated that the deficit of Nell-1 gene or loss NELL-1 function may contribute to the development of osteoporosis in animal and clinical researches. These studies suggest that the NELL-1 protein has potential to be used for treatment of osteoporosis by simple intravenous injection.

NELL-1 is often applied in local tissues (spine, femur, calvaria, etc) by being loaded onto various carriers including tricalcium phosphate (TCP) particles, demineralized bone matrix (DBM), and PLGA scaffold. But for the treatment of osteoporosis disease, it is necessary to be administered by intravenous injection that can lead to systemic functional improvement of bone quality. However, due to the rapid clearance of native protein drug in vivo, high dose and frequent administration usually have to be adopted to achieve therapeutic benefit. This can lead to high treatment cost and low patient compliance in chronic treatment. The short circulation time of NELL-1 in vivo could be one of the main limitations for the practical application of systemic therapy. Therefore, the main purpose of the present study was to extend the circulation time of NELL-1 in vivo by chemically modifying its molecular structure. Currently, one of the most popular technologies to prolong the half-life time of protein is to use water soluble polymers as a macromolecular carrier. As it is approved for human use by FDA, the non-toxic PEG molecule is widely used in numerous biomedical applications. It is a water soluble polymer with excellent biocompatibility but without immunogenicity. PEG is commercially available in a wide range of molecular weights, which is particularly appropriate for the chemical attachment to proteins with various molecular weights. So it was chosen to conjugate with NELL-1 protein in the current study.

The methods of chemical modification of protein with PEG can be achieved by site-specific conjugation. To the best of our knowledge, no reports have been made on the PEGylation of NELL-1, a huge protein with the Mw much larger than all other proteins that have been PEGylated to date. In one study of an embodiment of the present invention, we PEGylated NELL-1 by site-specific conjugation using three different PEG sizes (5, 20, 40 kDa). The PEGylated NELL-1 was synthesized using chemically activated PEG-N-hydroxysuccinimide (PEG-NHS) for conjugation with the amine group in lysine residue located at the surface of NELL-1. NHS was chosen for amine coupling reactions due to its high reactivity in bio-conjugation synthesis at physiological pH. For each PEGylated NELL-1, the PEG modification degree, thermal stability, and cytotoxicity were determined. The in vitro bioactivity study of NELL-PEG was also evaluated in two primary cell lines, human perivascular stem cells (hPSC) and mouse calvarial osteoblast cells. Subsequently, the pharmacokinetic behavior of the PEGylated NELL-1 was examined in mice.

### Bioconiugation to a Proteineous Drug

Chemical modification on a proteineous drug can be achieved through conjugation of a modifying group to the proteineous drug such as NELL-1 protein. Schemes (1)-(4) illustrate a few exemplary conjugation reactions to prepare and make conjugate (e.g., chemically modified NELL-1) by so-called click chemistry. See also, Craig S. McKayl and M. G. Finn, “Click Chemistry in Complex Mixtures: Bioorthogonal Bioconjugation” in Chemistry & Biology 21, Sep. 18, 2014, the teaching of which is incorporated herein in its entirety by reference.

Some examples of the modifying chemical group is an alkyl group, an aryl group, an acyl group, a leaving group, a polymer, a peptide, or a combination thereof.

Some further examples of the chemical group is poly(ethylene glycol) (PEG), poly(ethylene oxide) (PEO), poly(propylene glycol) (PPG), or poly(propylene oxide) (PPO).

Still some further examples of the chemical group is heparin sulfate (different from heparin), glycopolymers, zwitterionic polymers, hyperbranced polymers, polymers containing un-natural amino acids.

Bioconjugation to NELL-1 can be achieved via established reactions. For example, conjugation can occur via azide-alkyne, azide-BMCO, and Tetrazine-TCO type reactions. Other types of possible linkers to lysine, cysteine, etc. are described in Craig S. McKay 1 and M. G. Finn, 2014, supra, the teaching of which is incorporate herein by reference.

Some further examples of chemical group conjugation to NELL-1 include, for example, conjugating peptide sequences to Nell or to the conjugate to modulate interactions with ECM, target cells, immune cells, and hepatocytes, etc.

As further examples of conjugation to a proteineous drug (e.g., NELL-1 protein) include, for example, inserting responsive linkers that degrade on demand to external stimuli (pH, heat, specific wavelength, ultrasound, electric current, magnetic simulation, biomolecules and proteins). In such examples, one can allow a conjugate to circulate in blood in a protected form systemically, but at a selected site (e.g., the hip), he/she can stimulate the linker to degrade locally by delivering local stimuli for a desired period of time (e.g., 5 minutes a day, etc.) Alternatively, for spinal fusion, NELL-1 protein is protected as a conjugate and administered to the spinal fusion site and allowed to slowly diffuse until a stimuli is delivered to convert conjugate to NELL-1.

Still, as further examples, a natural enzyme is used to link a protective or biofunctionalized coating onto a proteineous drug (e.g., NELL). For example, one can use Factor XIII which crosslinks fibrinogen at specific sites. By encoding the Factor XIII peptide sequences into proteineous drug (e.g., NELL), or conjugating the peptides onto proteineous drug surface and the protective coating material, one can then use Factor XIII to conjugate the protective coating onto proteineous drug (e.g., NELL). Besides Factor XIII, many natural enzymes that act on natural proteins, and natural metabolic precursors can work. For example sortase A can be used by encoding or conjugating a short peptide sequence onto the proteineous drug (e.g., NELL).

### Method of Preparation

In another aspect of the present invention, it is provided a method of preparing a bone-targeting therapeutic conjugate of a formula of TG-M-D (I) or M-D-TG (II), comprising:

a) providing a therapeutic drug for a bone related condition,

b) providing a bone-targeting molecule,

c) providing an optional compound comprising a chemical group that modifies the therapeutic drug,

d) forming the conjugate,

wherein the conjugate is according to any one of any of the various embodiments disclosed herein.

In some embodiments of the invention method, optionally in combination with any one or all of the various embodiments disclosed herein, the bone targeting molecule is a bisphosphate or DSS peptide.

In some embodiments of the invention method, optionally in combination with any one or all of the various embodiments disclosed herein, the bone targeting molecule is a bisphosphonate comprising a chemical formula:

(OX)₂(O)P—(CR₁R₂)ₙ—P(O)(OM)₂, wherein:

each X is independently H, or a cation,

each R₁ is independently a hydroxyl, amino, thiol, amide, or carboxyl group,

each R₂ is a C1-C20 group comprising optional hetero atom(s), and

n is an integer ranging from 1-10, e.g., 1, 2, 3, 4, 5, 6, 7, 8, 9 or 10.

In some embodiments of the invention method, optionally in combination with any one or all of the various embodiments disclosed herein, the bisphosphonate is one of:

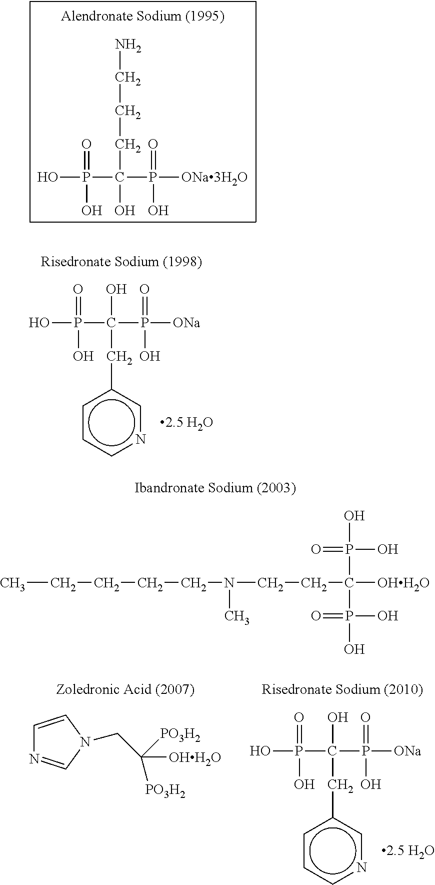

In some embodiments of the invention method, optionally in combination with any one or all of the various embodiments disclosed herein, the therapeutic drug is a small molecule drug.

In some embodiments of the invention conjugate, optionally in combination with any one or all of the various embodiments disclosed herein, the therapeutic drug is a proteineous drug.

In some embodiments of the invention method, optionally in combination with any one or all of the various embodiments disclosed herein, the proteineous drug is NELL-1 protein or a BMP protein.

In some embodiments of the invention method, optionally in combination with any one or all of the various embodiments disclosed herein, the optional chemical group comprises an alkyl group, an aryl group, an acyl group, a leaving group, a polymer, a peptide, or a combination thereof.

In some embodiments of the invention method, optionally in combination with any one or all of the various embodiments disclosed herein, the optional chemical group is poly(ethylene glycol) (PEG), poly(ethylene oxide) (PEO), poly(propylene glycol) (PPG), or poly(propylene oxide) (PPO).

In some embodiments of the invention method, optionally in combination with any one or all of the various embodiments disclosed herein, the optional chemical group is selected from heparin sulfate, glycopolymers, zwitterionic polymers, hyperbranced polymers, polymers containing unnatural amino acids, linkers to lysine or cysteine, or peptide sequences that modify drug or conjugateinteractions with ECM, target cells, immune cells, and hepatocytes.

In some embodiments of the invention method, optionally in combination with any one or all of the various embodiments disclosed herein, the optional chemical group comprises a responsive linker that degrades on demand to external stimuli.

In some embodiments of the invention method, optionally in combination with any one or all of the various embodiments disclosed herein, the optional chemical group provides a linking reaction that is azide-alkyne, azide-BMCO, or Tetrazine-TCO type reactions.

In some embodiments of the invention method, optionally in combination with any one or all of the various embodiments disclosed herein, the optional chemical group comprises a linker to lysine or cysteine.

In some embodiments of the invention method, optionally in combination with any one or all of the various embodiments disclosed herein, the optional chemical group comprises a natural enzyme.

In some embodiments of the invention method, optionally in combination with any or all of the various embodiments disclosed herein, the at least one chemical group imparts at least one desirable property to the therapeutic drug such that the conjugate is significantly improved in the at least one desirable property relative to a naked therapeutic drug (e.g., NELL-1 protein) without chemical modification.

In some embodiments of the invention method, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

### Compositions

In another aspect of the present invention, it is provided a composition, comprising a bone-targeting therapeutic conjugate according to any of the various embodiments disclosed herein.

In some embodiments of the invention composition, optionally in combination with any one or all of the various embodiments disclosed herein, the composition further comprises a pharmaceutically acceptable carrier.

In some embodiments of the invention composition, optionally in combination with any one or all of the various embodiments disclosed herein, the bone targeting molecule is a bisphosphate or DSS peptide.

In some embodiments of the invention composition, optionally in combination with any one or all of the various embodiments disclosed herein, the bone targeting molecule is a bisphosphonate comprising a chemical formula:

(OX)₂(O)P—(CR₁R₂)ₙ—P(O)(OM)₂, wherein:

each X is independently H, or a cation,

each R₁ is independently a hydroxyl, amino, thiol, amide, or carboxyl group,

each R₂ is a C1-C20 group comprising optional hetero atom(s), and

n is an integer ranging from 1-10, e.g., 1, 2, 3, 4, 5, 6, 7, 8, 9 or 10.

In some embodiments of the invention composition, optionally in combination with any one or all of the various embodiments disclosed herein, the bisphosphonate is one of:

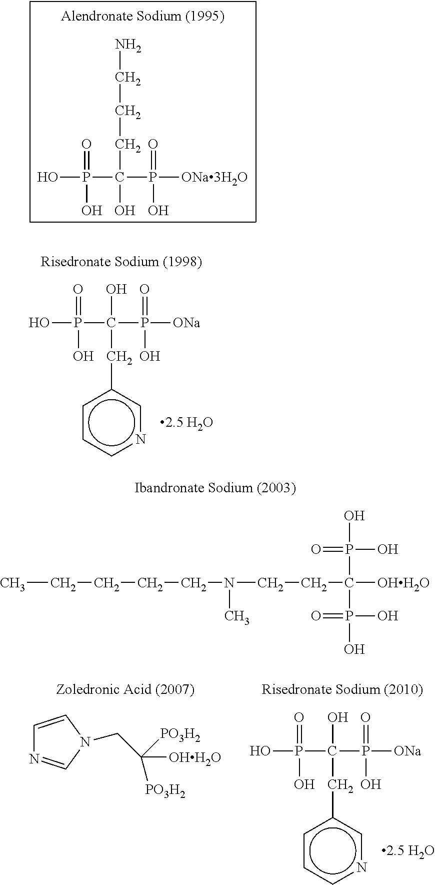

In some embodiments of the invention composition, optionally in combination with any one or all of the various embodiments disclosed herein, the therapeutic drug is a small molecule drug.

In some embodiments of the invention composition, optionally in combination with any one or all of the various embodiments disclosed herein, the therapeutic drug is a proteineous drug.

In some embodiments of the invention composition, optionally in combination with any one or all of the various embodiments disclosed herein, the proteineous drug is NELL-1 protein or a BMP protein.

In some embodiments of the invention composition, optionally in combination with any one or all of the various embodiments disclosed herein, the optional chemical group comprises an alkyl group, an aryl group, an acyl group, a leaving group, a polymer, a peptide, or a combination thereof.

In some embodiments of the invention composition, optionally in combination with any one or all of the various embodiments disclosed herein, the optional chemical group is poly(ethylene glycol) (PEG), poly(ethylene oxide) (PEO), poly(propylene glycol) (PPG), or poly(propylene oxide) (PPO).

In some embodiments of the invention composition, optionally in combination with any one or all of the various embodiments disclosed herein, the optional chemical group is selected from heparin sulfate, glycopolymers, zwitterionic polymers, hyperbranced polymers, polymers containing unnatural amino acids, linkers to lysine or cysteine, or peptide sequences that modify drug or conjugateinteractions with ECM, target cells, immune cells, and hepatocytes.

In some embodiments of the invention composition, optionally in combination with any one or all of the various embodiments disclosed herein, the optional chemical group comprises a responsive linker that degrades on demand to external stimuli.

In some embodiments of the invention composition, optionally in combination with any one or all of the various embodiments disclosed herein, the optional chemical group provides a linking reaction that is azide-alkyne, azide-BMCO, or Tetrazine-TCO type reactions.

In some embodiments of the invention composition, optionally in combination with any one or all of the various embodiments disclosed herein, the optional chemical group comprises a linker to lysine or cysteine.

In some embodiments of the invention composition, optionally in combination with any one or all of the various embodiments disclosed herein, the optional chemical group comprises a natural enzyme.

In some embodiments of the invention composition, optionally in combination with any or all of the various embodiments disclosed herein, the composition is a formulation for systemic or local delivery.

In some embodiments of the invention composition, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

### Methods of Fabrication

In another aspect of the present invention, it is provided a method of forming a composition, comprising

providing an amount of a bone-targeting therapeutic conjugate of a formula of TG-M-D (I) or M-D-TG (II), and

forming the composition,

wherein the conjugate is according to any one of any of the various embodiments disclosed herein.

In some embodiments of the invention method, optionally in combination with any one or all of the various embodiments disclosed herein, the composition further comprises a pharmaceutically acceptable carrier.

In some embodiments of the invention method, optionally in combination with any one or all of the various embodiments disclosed herein, the bone targeting molecule is a bisphosphate or DSS peptide.

In some embodiments of the invention method, optionally in combination with any one or all of the various embodiments disclosed herein, the bone targeting molecule is a bisphosphonate comprising a chemical formula:

(OX)₂(O)P—(CR₁R₂)ₙ—P(O)(OM)₂, wherein:

each X is independently H, or a cation,

each R₁ is independently a hydroxyl, amino, thiol, amide, or carboxyl group,

each R₂ is a C1-C20 group comprising optional hetero atom(s), and

n is an integer ranging from 1-10, e.g., 1, 2, 3, 4, 5, 6, 7, 8, 9 or 10.

In some embodiments of the invention method, optionally in combination with any one or all of the various embodiments disclosed herein, the bisphosphonate is one of:

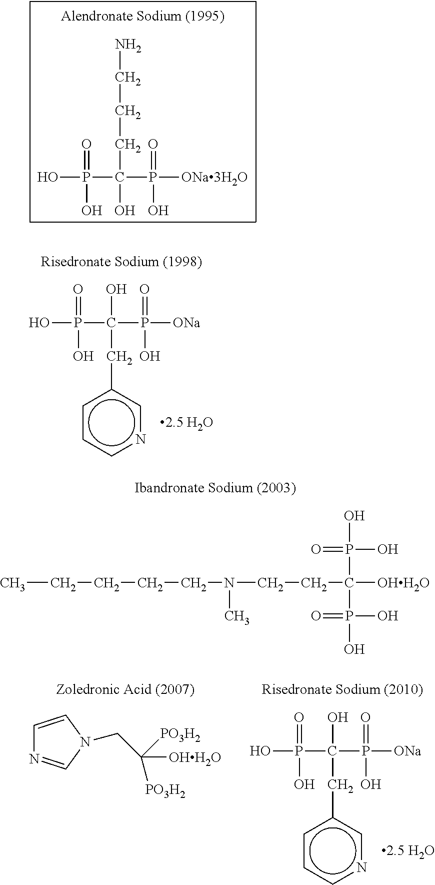

In some embodiments of the invention method, optionally in combination with any one or all of the various embodiments disclosed herein, the therapeutic drug is a small molecule drug.

In some embodiments of the invention method, optionally in combination with any one or all of the various embodiments disclosed herein, the therapeutic drug is a proteineous drug.

In some embodiments of the invention method, optionally in combination with any one or all of the various embodiments disclosed herein, the proteineous drug is NELL-1 protein or a BMP protein.

In some embodiments of the invention method, optionally in combination with any one or all of the various embodiments disclosed herein, the optional chemical group comprises an alkyl group, an aryl group, an acyl group, a leaving group, a polymer, a peptide, or a combination thereof.

In some embodiments of the invention method, optionally in combination with any one or all of the various embodiments disclosed herein, the optional chemical group is poly(ethylene glycol) (PEG), poly(ethylene oxide) (PEO), poly(propylene glycol) (PPG), or poly(propylene oxide) (PPO).

In some embodiments of the invention method, optionally in combination with any one or all of the various embodiments disclosed herein, the optional chemical group is selected from heparin sulfate, glycopolymers, zwitterionic polymers, hyperbranced polymers, polymers containing unnatural amino acids, linkers to lysine or cysteine, or peptide sequences that modify drug or conjugateinteractions with ECM, target cells, immune cells, and hepatocytes.

In some embodiments of the invention method, optionally in combination with any one or all of the various embodiments disclosed herein, the optional chemical group comprises a responsive linker that degrades on demand to external stimuli.

In some embodiments of the invention method, optionally in combination with any one or all of the various embodiments disclosed herein, the optional chemical group provides a linking reaction that is azide-alkyne, azide-BMCO, or Tetrazine-TCO type reactions.

In some embodiments of the invention method, optionally in combination with any one or all of the various embodiments disclosed herein, the optional chemical group comprises a linker to lysine or cysteine.

In some embodiments of the invention method, optionally in combination with any one or all of the various embodiments disclosed herein, the optional chemical group comprises a natural enzyme.

In some embodiments of the invention method, optionally in combination with any or all of the various embodiments disclosed herein, the composition is a formulation for systemic or local delivery.

In some embodiments of the invention method, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

### Carriers

The present invention involves compositions useful for practicing the therapeutic methods described herein. Therapeutic compositions contain a physiologically tolerable carrier together with an active agent as described herein, dissolved or dispersed therein as an active ingredient. In a preferred embodiment, the therapeutic composition is not immunogenic when administered to a mammal or human patient for therapeutic purposes. As used herein, the terms “pharmaceutically acceptable”, “physiologically tolerable” and grammatical variations thereof, as they refer to compositions, carriers, diluents and reagents, are used interchangeably and represent that the materials are capable of administration to or upon a mammal without the production of undesirable physiological effects such as nausea, dizziness, gastric upset and the like. A pharmaceutically acceptable carrier will not promote the raising of an immune response to an agent with which it is admixed, unless so desired. The preparation of a pharmacological composition that contains active ingredients dissolved or dispersed therein is well understood in the art and need not be limited based on formulation. Typically such compositions are prepared as injectable either as liquid solutions or suspensions, however, solid forms suitable for solution, or suspensions, in liquid prior to use can also be prepared. The preparation can also be emulsified or presented as a liposome composition. The active ingredient can be mixed with excipients which are pharmaceutically acceptable and compatible with the active ingredient and in amounts suitable for use in the therapeutic methods described herein. Suitable excipients include, for example, water, saline, dextrose, glycerol, ethanol or the like and combinations thereof. In addition, if desired, the composition can contain minor amounts of auxiliary substances such as wetting or emulsifying agents, pH buffering agents and the like which enhance the effectiveness of the active ingredient. The therapeutic composition of the present invention can include pharmaceutically acceptable salts of the components therein. Pharmaceutically acceptable salts include the acid addition salts that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Physiologically tolerable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent used in the methods described herein that will be effective in the treatment of a particular disorder or condition will depend on the nature of the disorder or condition, and can be determined by standard clinical techniques.

Pharmaceutically acceptable carrier is well known in the art. Examples of such carrier includes, e.g., salient, for liquid or suspension formulations, natural or synthetic polymeric materials for burst or sustained release formulations or targeted delivery formulations. Some examples of the carriers are further described in detail below.

### Polymeric Materials

In some embodiments, the carrier disclosed herein can be a polymeric material Exemplary polymeric material that can be used here include but are not limited to a biocompatible or bioabsorbable polymer that is one or more of poly(DL-lactide), poly(L-lactide), poly(L-lactide), poly(L-lactide-co-DL-lactide), polymandelide, polyglycolide, poly(lactide-co-glycolide), poly(D,L-lactide-co-glycolide), poly(L-lactide-co-glycolide), poly(ester amide), poly(ortho esters), poly(glycolic acid-co-trimethylene carbonate), poly(D,L-lactide-co-trimethylene carbonate), poly(trimethylene carbonate), poly(lactide-co-caprolactone), poly(glycolide-co-caprolactone), poly(tyrosine ester), polyanhydride, derivatives thereof. In some embodiments, the polymeric material comprises a combination of these polymers.

In some embodiments, the polymeric material comprises poly(D,L-lactide-co-glycolide). In some embodiments, the polymeric material comprises poly(D,L-lactide). In some embodiments, the polymeric material comprises poly(L-lactide). [0065] Additional exemplary polymers include but are not limited to poly(D-lactide) (PDLA), polymandelide (PM), polyglycolide (PGA), poly(L-lactide-co-D,L-lactide) (PLDLA), poly(D,L-lactide) (PDLLA), poly(D,L-lactide-co-glycolide) (PLGA) and poly(L-lactide-co-glycolide) (PLLGA). With respect to PLLGA, the stent scaffolding can be made from PLLGA with a 25 mole % of GA between 5-15 mol %. The PLLGA can have a mole % of (LA:GA) of 85:15 (or a range of 82:18 to 88:12), 95:5 (or a range of 93:7 to 97:3), or commercially available PLLGA products identified as being 85:15 or 95:5 PLLGA. The examples provided above are not the only polymers that may be used. Many other examples can be provided, such as those found in Polymeric Biomaterials, second edition, edited by Severian Dumitriu; chapter 4.

In some embodiments, polymers that are more flexible or that have a lower modulus that those mentioned above may also be used. Exemplary lower modulus bioabsorbable polymers include, polycaprolactone (PCL), poly(trimethylene carbonate) (PTMC), polydioxanone (PDO), poly(3-hydrobutyrate) (PHB), poly(4-hydroxybutyrate) (P4HB), poly(hydroxyalkanoate) (PHA), and poly(butylene succinate), and blends and copolymers thereof.

In exemplary embodiments, higher modulus polymers such as PLLA or PLLGA may be blended with lower modulus polymers or copolymers with PLLA or PLGA. The blended lower modulus polymers result in a blend that has a higher fracture toughness than the high modulus polymer. Exemplary low modulus copolymers include poly(L-lactide)-b-polycaprolactone (PLLA-b-PCL) or poly(L-lactide)-co-polycaprolactone (PLLA-co-PCL). The composition of a blend can include 1-5 wt % of low modulus polymer.

More exemplary polymers include but are not limited to at least partially alkylated polyethyleneimine (PEI); at least partially alkylated poly(lysine); at least partially alkylated polyornithine; at least partially alkylated poly(amido amine), at least partially alkylated homo- and co-polymers of vinylamine; at least partially alkylated acrylate containing aminogroups, copolymers of vinylamine containing aminogroups with hydrophobic monomers, copolymers of acrylate containing aminogroups with hydrophobic monomers, and amino containing natural and modified polysaccharides, polyacrylates, polymethacryates, polyureas, polyurethanes, polyolefins, polyvinylhalides, polyvinylidenehalides, polyvinylethers, polyvinylaromatics, polyvinylesters, polyacrylonitriles, alkyd resins, polysiloxanes and epoxy resins, and mixtures thereof [0069] Additional examples of biocompatible biodegradable polymers include, without limitation, polycaprolactone, poly(L-lactide), poly(D,L-lactide), poly(D,L-lactide-co-PEG) block copolymers, poly(D,L-lactide-co-trimethylene carbonate), poly(lactide-co-glycolide), polydioxanone (PDS), polyorthoester, polyanhydride, poly(glycolic acid-co-trimethylene carbonate), polyphosphoester, polyphosphoester urethane, poly(amino acids), polycyanoacrylates, poly(trimethylene carbonate), poly(iminocarbonate), polycarbonates, polyurethanes, polyalkylene oxalates, polyphosphazenes, PHA-PEG, and combinations thereof. The PHA may include poly(a-hydroxyacids), poly(P-hydroxyacid) such as poly(3-hydroxybutyrate) (PHB), poly(3-hydroxybutyrate-co-valerate) (PHBV), poly(3-hydroxyproprionate) (PHP), poly(3-hydroxyhexanoate) (PHH), or poly(4-hydroxyacid) such as poly poly(4-hydroxybutyrate), poly(4-hydroxyvalerate), poly(4-hydroxyhexanoate), poly(hydroxyvalerate), poly(tyrosine carbonates), poly(tyrosine arylates), poly(ester amide), polyhydroxyalkanoates (PHA), poly(3-hydroxyalkanoates) such as poly(3-hydroxypropanoate), poly(3-hydroxybutyrate), poly(3-hydroxyvalerate), poly(3-hydroxyhexanoate), poly(3-hydroxyheptanoate) and poly(3-hydroxyoctanoate), poly(4-hydroxyalkanaote) such as poly(4-hydroxybutyrate), poly(4-hydroxyvalerate), poly(4-hydroxyhexanote), poly(4-hydroxyheptanoate), poly(4-hydroxyoctanoate) and copolymers including any of the 3-hydroxyalkanoate or 4-hydroxyalkanoate monomers described herein or blends thereof, poly(D,L-lactide), poly(L-lactide), polyglycolide, poly(D,L-lactide-co-glycolide), poly(L-lactide-co-glycolide), polycaprolactone, poly(lactide-co-caprolactone), poly(glycolide-co-caprolactone), poly(dioxanone), poly(ortho esters), poly(anhydrides), poly(tyrosine carbonates) and derivatives thereof, poly(tyrosine ester) and derivatives thereof, poly(imino carbonates), poly(glycolic acid-co-trimethylene carbonate), polyphosphoester, polyphosphoester urethane, poly(amino acids), polycyanoacrylates, poly(trimethylene carbonate), poly(iminocarbonate), polyphosphazenes, silicones, polyesters, polyolefins, polyisobutylene and ethylene-alphaolefin copolymers, acrylic polymers and copolymers, vinyl halide polymers and copolymers, such as polyvinyl chloride, polyvinyl ethers, such as polyvinyl methyl ether, polyvinylidene halides, such as polyvinylidene chloride, polyacrylonitrile, polyvinyl ketones, polyvinyl aromatics, such as polystyrene, polyvinyl esters, such as polyvinyl acetate, copolymers of vinyl monomers with each other and olefins, such as ethylene-methyl methacrylate copolymers, acrylonitrile-styrene copolymers, ABS resins, and ethylene-vinyl acetate copolymers, polyamides, such as Nylon 66 and polycaprolactam, alkyd resins, polycarbonates, polyoxymethylenes, polyimides, polyethers, poly(glyceryl sebacate), poly(propylene fumarate), poly(n-butyl methacrylate), poly(sec-butyl methacrylate), poly(isobutyl methacrylate), poly(tert-butyl methacrylate), poly(n-propyl methacrylate), poly(isopropyl methacrylate), poly(ethyl methacrylate), poly(methyl methacrylate), epoxy resins, polyurethanes, rayon, rayon-triacetate, cellulose acetate, cellulose butyrate, cellulose acetate butyrate, cellophane, cellulose nitrate, cellulose propionate, cellulose ethers, carboxymethyl cellulose, polyethers such as poly(ethylene glycol) (PEG), copoly(ether-esters) (e.g. poly(ethylene oxide-co-lactic acid) (PEO/PLA)), polyalkylene oxides such as poly(ethylene oxide), poly(propylene oxide), poly(ether ester), polyalkylene oxalates, phosphoryl choline containing polymer, choline, poly(aspirin), polymers and co-polymers of hydroxyl bearing monomers such as 2-hydroxyethyl methacrylate (HEMA), hydroxypropyl methacrylate (HPMA), hydroxypropylmethacrylamide, PEG acrylate (PEGA), PEG methacrylate, methacrylate polymers containing 2-methacryloyloxyethyl-phosphorylcholine (MPC) and n-vinyl pyrrolidone (VP), carboxylic acid bearing monomers such as methacrylic acid (MA), acrylic acid (AA), alkoxymethacrylate, alkoxyacrylate, and 3-trimethylsilylpropyl methacrylate (TMSPMA), poly(styrene-isoprene-styrene)-PEG (SIS-PEG), polystyrene-PEG, polyisobutylene-PEG, polycaprolactone-PEG (PCL-PEG), PLA-PEG, poly(methyl methacrylate), MED610, poly(methyl methacrylate)-PEG (PMMA-PEG), polydimethylsiloxane-co-PEG (PDMS-PEG), poly(vinylidene fluoride)-PEG (PVDF-PEG), PLURONIC™ surfactants (polypropylene oxide-co-polyethylene glycol), poly(tetramethylene glycol), hydroxy functional poly(vinyl pyrrolidone), biomolecules such as collagen, chitosan, alginate, fibrin, fibrinogen, cellulose, starch, dextran, dextrin, hyaluronic acid, fragments and derivatives of hyaluronic acid, heparin, fragments and derivatives of heparin, glycosamino glycan (GAG), GAG derivatives, polysaccharide, elastin, elastin protein mimetics, or combinations thereof.

In some embodiments, polyethylene is used to construct at least a portion of the device. For example, polyethylene can be used in an orthopedic implant on a surface that is designed to contact another implant, as such in a joint or hip replacement. Polyethylene is very durable when it comes into contact with other materials. When a metal implant moves on a polyethylene surface, as it does in most joint replacements, the contact is very smooth and the amount of wear is minimal. Patients who are younger or more active may benefit from polyethylene with even more resistance to wear. This can be accomplished through a process called crosslinking, which creates stronger bonds between the elements that make up the polyethylene. The appropriate amount of crosslinking depends on the type of implant. For example, the surface of a hip implant may require a different degree of crosslinking than the surface of a knee implant.

Additional examples of polymeric materials can be found, for example, in U.S. Pat. No. 6,127,448 to Domb, US Pat. Pub. No. 2004/0148016 by Klein and Brazil, US Pat. Pub. No. 2009/0169714 by Burghard et al., U.S. Pat. No. 6,406,792 to Briquet et al, US Pat. Pub. No. 2008/0003256 by Martens et al, each of which is hereby incorporated by reference herein in its entirety.

### Methods of Use

In another aspect of the present invention, it is provided a method of treating or ameliorating a condition in a subject, comprising administering to the subject a bone-targeting therapeutic conjugate of a formula of TG-M-D (I) or M-D-TG (II),

wherein the conjugate is according to any one of any of the various embodiments disclosed herein.

In some embodiments of the invention method, optionally in combination with any one or all of the various embodiments disclosed herein, the bone targeting molecule is a bisphosphate or DSS peptide.

In some embodiments of the invention method, optionally in combination with any one or all of the various embodiments disclosed herein, the bone targeting molecule is a bisphosphonate comprising a chemical formula:

(OX)₂(O)P—(CR₁R₂)ₙ—P(O)(OM)₂, wherein:

each X is independently H, or a cation,

each R₁ is independently a hydroxyl, amino, thiol, amide, or carboxyl group,

each R₂ is a C1-C20 group comprising optional hetero atom(s), and

n is an interger ranging from 1-10, e.g., 1, 2, 3, 4, 5, 6, 7, 8, 9 or 10.

In some embodiments of the invention method, optionally in combination with any one or all of the various embodiments disclosed herein, the bisphosphonate is one of:

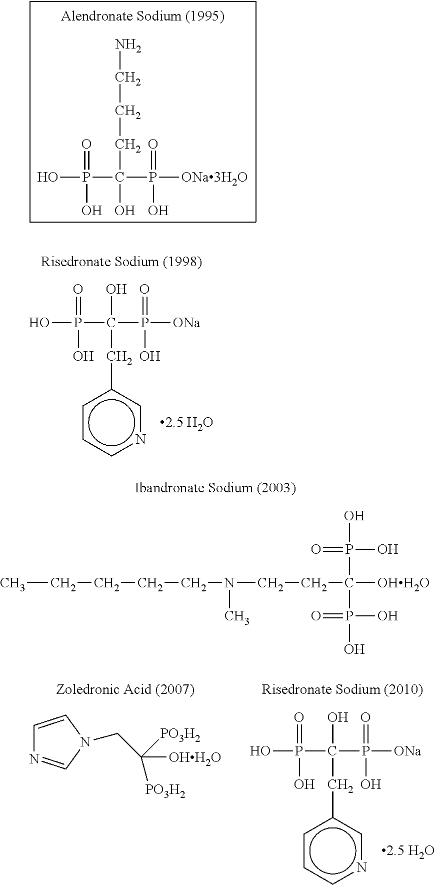

In some embodiments of the invention method, optionally in combination with any one or all of the various embodiments disclosed herein, the therapeutic drug is a small molecule drug.

In some embodiments of the invention method, optionally in combination with any one or all of the various embodiments disclosed herein, the therapeutic drug is a proteineous drug.

In some embodiments of the invention method, optionally in combination with any one or all of the various embodiments disclosed herein, the proteineous drug is NELL-1 protein or a BMP protein.

In some embodiments of the invention method, optionally in combination with any one or all of the various embodiments disclosed herein, the optional chemical group comprises an alkyl group, an aryl group, an acyl group, a leaving group, a polymer, a peptide, or a combination thereof.

In some embodiments of the invention method, optionally in combination with any one or all of the various embodiments disclosed herein, the optional chemical group is poly(ethylene glycol) (PEG), poly(ethylene oxide) (PEO), poly(propylene glycol) (PPG), or poly(propylene oxide) (PPO).

In some embodiments of the invention method, optionally in combination with any one or all of the various embodiments disclosed herein, the optional chemical group is selected from heparin sulfate, glycopolymers, zwitterionic polymers, hyperbranced polymers, polymers containing unnatural amino acids, linkers to lysine or cysteine, or peptide sequences that modify drug or conjugateinteractions with ECM, target cells, immune cells, and hepatocytes.

In some embodiments of the invention method, optionally in combination with any one or all of the various embodiments disclosed herein, the optional chemical group comprises a responsive linker that degrades on demand to external stimuli.

In some embodiments of the invention method, optionally in combination with any one or all of the various embodiments disclosed herein, the optional chemical group provides a linking reaction that is azide-alkyne, azide-BMCO, or Tetrazine-TCO type reactions.

In some embodiments of the invention method, optionally in combination with any one or all of the various embodiments disclosed herein, the optional chemical group comprises a linker to lysine or cysteine.

In some embodiments of the invention method, optionally in combination with any one or all of the various embodiments disclosed herein, the optional chemical group comprises a natural enzyme.

In some embodiments of the invention method, optionally in combination with any or all of the various embodiments disclosed herein, the composition is a formulation for systemic or local delivery.

In some embodiments of the invention method, optionally in combination with any or all of the various embodiments of the present invention, the at least one desirable property is selected from the group consisting of blood circulation life, shelf-life, hydrophobicity or hydrophilicity, biological activity, bioavailability, cytotoxicity, non-immunogenicity, or conformational properties, etc.

In some embodiments of the invention method, optionally in combination with any one or all of the various embodiments disclosed herein, the conjugate is included in a composition that comprises the conjugate and a pharmaceutically acceptable carrier.

In some embodiments of the invention method, optionally in combination with any one or all of the various embodiments disclosed herein, the bone related condition is osteoporosis.

In some embodiments of the invention method, optionally in combination with any one or all of the various embodiments disclosed herein, the bone related condition is bone fracture or intervertebral disc disease or injury.

In some embodiments of the invention method, optionally in combination with any one or all of the various embodiments disclosed herein, administering comprises local or systemic administration.

In some embodiments of the invention method, optionally in combination with any one or all of the various embodiments disclosed herein, the subject is a human being.

### Dosage and Administration

The dosage can be determined by one of skill in the art and can also be adjusted by the individual physician in the event of any complication. Typically, the dosage ranges from 0.0005 mg/kg body weight to 1 g/kg body weight. In some embodiments, the dosage range is from 0.001 mg/kg body weight to 0.5 g/kg body weight, from 0.0005 mg/kg body weight to 0.1 g/kg body weight, from 0.001 mg/kg body weight to 0.05 g/kg body weight.

As another alternative, dosage is selected for localized delivery and is not necessary selected to body weight or to achieve a certain serum level, but to achieve a localized effect, e.g., as for a localized injection, implantation or other localized administration to the eye.

Administration of the doses recited above can be repeated for a limited period of time. In some embodiments, the doses are given once a day, or multiple times a day, for example but not limited to three times a day. In a preferred embodiment, the doses recited above are administered daily for several weeks or months. The duration of treatment depends upon the subject's clinical progress and responsiveness to therapy. Continuous, relatively low maintenance doses are contemplated after an initial higher therapeutic dose.

Agents useful in the methods and compositions described herein can be administered topically, intravenously (by bolus or continuous infusion), orally, by inhalation, intraperitoneally, intramuscularly, subcutaneously, intracavity, and can be delivered by peristaltic means, if desired, or by other means known by those skilled in the art. It is preferred that the agents for the methods described herein are administered topically to the eye. For the treatment of tumors, the agent can be administered systemically, or alternatively, can be administered directly to the tumor e.g., by intratumor injection or by injection into the tumor's primary blood supply.

Therapeutic compositions containing at least one agent disclosed herein can be conventionally administered in a unit dose. The term “unit dose” when used in reference to a therapeutic composition refers to physically discrete units suitable as unitary dosage for the subject, each unit containing a predetermined quantity of active material calculated to produce the desired therapeutic effect in association with the required physiologically acceptable diluent, i.e., carrier, or vehicle.

The compositions are administered in a manner compatible with the dosage formulation, and in a therapeutically effective amount. The quantity to be administered and timing depends on the subject to be treated, capacity of the subject's system to utilize the active ingredient, and degree of therapeutic effect desired. An agent can be targeted by means of a targeting moiety, such as e.g., an antibody or targeted liposome technology. Antibody-based or non-antibody-based targeting moieties can be employed to deliver a ligand or the inhibitor to a target site. Preferably, a natural binding agent for an unregulated or disease associated antigen is used for this purpose.

Precise amounts of active ingredient required to be administered depend on the judgment of the practitioner and are particular to each individual. However, suitable dosage ranges for systemic application are disclosed herein and depend on the route of administration. Suitable regimes for administration are also variable, but are typified by an initial administration followed by repeated doses at one or more intervals by a subsequent injection or other administration. Alternatively, continuous intravenous infusion sufficient to maintain concentrations in the blood in the ranges specified for in vivo therapies are contemplated.

An agent may be adapted for catheter-based delivery systems including coated balloons, slow-release drug-eluting stents or other drug-eluting formats, microencapsulated PEG liposomes, or nanobeads for delivery using direct mechanical intervention with or without adjunctive techniques such as ultrasound.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The following examples illustrate rather than limit the embodiments of the present invention.

### EXAMPLE 1

### Studies on DSS or Bisphosphonate PEG-NELL-1 Conjugates

### Summary of the Studies

The study was carried out with the aim of developing a novel protein based therapeutic agents for osteoporosis. As the results shown below, onjugating the PEG-NELL-1 with a bisphosphonate that exhibit strong bone affinity allows one to target the protein to bone tissue after systemic administration.

Due to lack of direct osteoblast-specific targeting system, the bisphosphonate (BP) was used in this project. The BP can guide PEG-NELL protein bonding to hydroxyapatite (HA) in bone tissue. The binding between them is reversible, and the BP-PEG-NELL will slowly dissociate from HA when its concentration in extracellular fluid going down, which lead to a sustained release followed by a long-term biological effect to stimulate bone regeneration.

The HA in bone will work as a reservoir of BP-PEG-NELL, providing growth factor to nearby osteoblast and osteoclast.

Since the PEG-NELL increases bone strength primarily by stimulating bone formation, whereas BP can reduce bone resorption, the conjugate would increase bone density more than the use of either one alone; (Otherwise, if the dose of BP is below the minimum effective dose, the BP will only work as a targeting molecule).

### Biphosphonate Approved by FDA

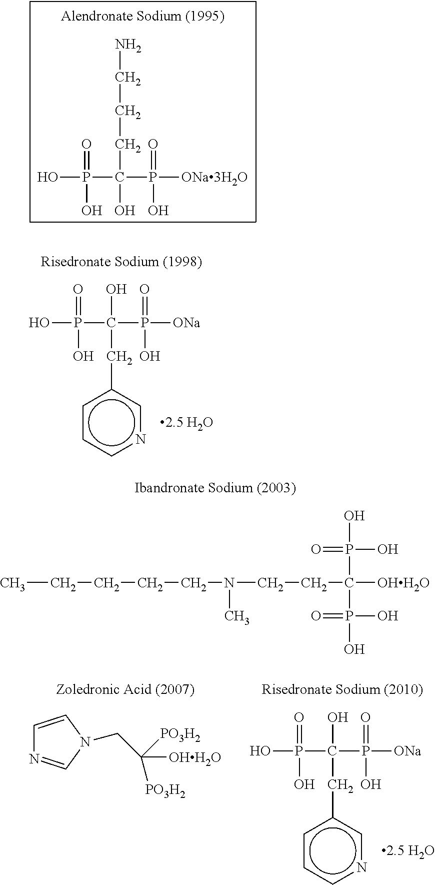

In the studies described here, alendronate sodium was used. The reasons for using alendronate sodium are:

a) amine group can be used for conjugation; and

b) the dose can be used is higher than other drugs (10 mg daily and 70 mg weekly, low toxicity).

Comparison of targeting NELL conjugate is summarized below (Table 1)

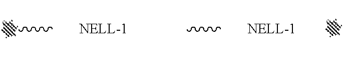

TABLE 1

| | DSS-PEG-NELL | BP-PEG-NELL |
|---|---|---|
| Structure | DSS conjugate to PEG and then | BP and PEG |
| | connect to NELL-1 | conjugate to NELL |
| Target | [symbol] DSS, repeat of | [symbol] BP, chemical drug, |
| molecule | tripeptide | FDA approved |
| Binding | HA, especially lower | HA |
| site | crystallinity site | |
| Biological | No activity itself, does | |
| effect | not affect the effect | |
| | of therapeutic agents | |
| Safety | Stem from intrinsic | |
| | protein (salivary protein, | |
| | osteocalcin, etc), quite safe | |

BP-PEG-NELL synthesis

An example conjugate BP-PEG-NELL-1 was synthesized according to Scheme 1.

### DSS-PEG-NELL Synthesis

Synthesis of DSS-PEG-NELL-1 was carried out according to Scheme 1.

### Determination of Modification Degree

Degree of modification of DSS-PEG-NELL was determined by fluorometric assay using fluorescamine and summarized in Table 2.

TABLE 2

| | Available | | | |
|---|---|---|---|---|
| | group | Modified | Target molecule | |
| | amount | group/molecule | amount/molecule | Structure |
| BP-PEG-NELL | 43 | 19 | 3 | 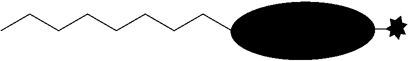 |
| DSS-PEG-NELL | 43 | 25 | 25 | 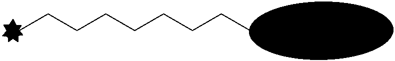 |

### HA Binding Test (The Affinity of Conjugates)

HA binding tests were conducted under the conditions below. The results are shown in FIG. 1.

### Condition:

0.5 mg of HA powder(NanoXIM HA, 5±1 μm d₅₀), 10 μg of protein, incubate for 1 h under shaking

### Conclusion:

After conjugated with targeting molecule, more protein can be absorbed onto the HA powder. BP vs DSS, PEG interference.

Comparison Studies on the Thermal Stability of Native NELL-1 with NELL-1 Conjuages

### Results:

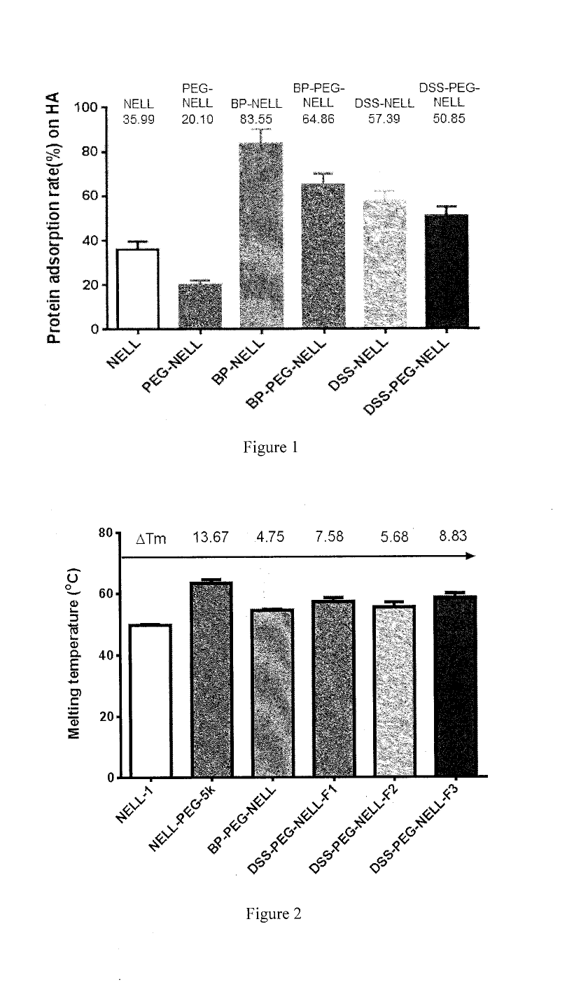

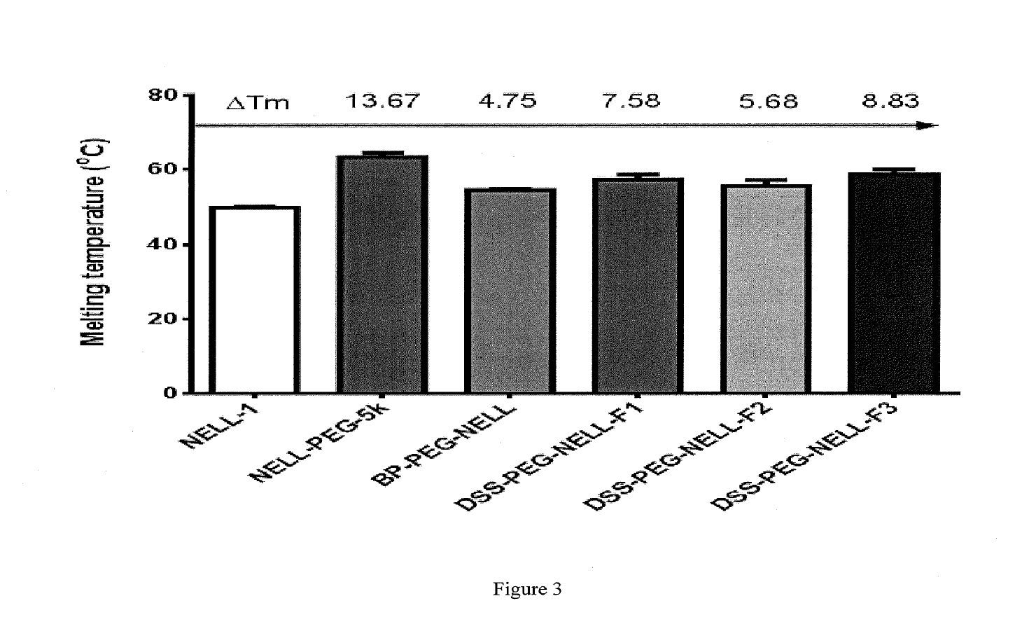

Studies on the thermal stability of native NELL-1 and different targeting NELL-1 conjuages of invention were performed (FIGS. 2 and 3). Comparison of the thermal stability test results is summarized in in FIG. 2, which shows that the difference between DSS-NELL and DSS-PEG-NELL in the thermal stability is smaller than BP-NELL and BP-PEG-NELL. FIG. 3 summarizes the test resutls on thermal stability of different targeting NELL-1 conjuages of invention.

### Conclusion:

Compared to native NELL-1, the thermal stability of BP-PEG-NELL and DSS-PEG-NELL is much higher, although they are smaller than the PEG-NELL-1.

### Biodistribution Studies

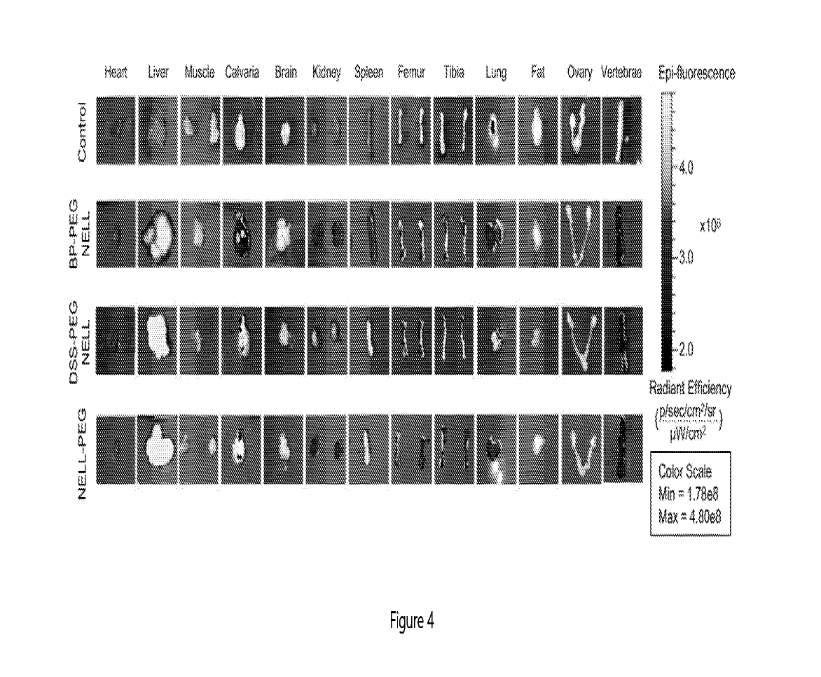

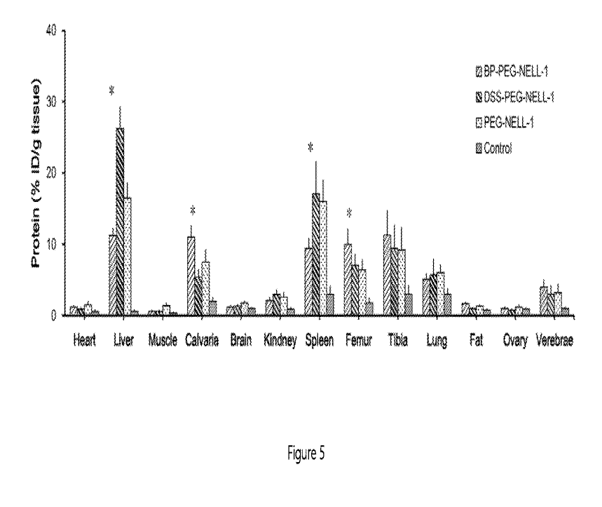

Biodistribution studies were performed. The results are summarized in FIGS. 4 and 5. FIG. 4 shows biodistribution at time-point 48 h. Overall, FIGS. 4 and 5 show:
1) The bisphosphonate significantly changed the biodistribution of PEG-NELL protein—
   a) less protein was distributed into liver, spleen, and other organs;
   b) more protein was distributed into the calvaria, femur, tibia, vertebrae, and the bone tissue;
   c) the distribution in heart, brain remains in low level.
2) The DSS did not change too much on the distribution pattern as BP did, but DSS appears to be a safer targeting molecule (data not shown).

### Studies on the Bioactivity of the BP-PEG-NELL, DSS-PEG-NELL In Vitro

### Materials and Procedures

Materials: hPSC and NMCC (pro-osteoblast) cells,

ALP testing and alizarin red staining

In vitro and in vivo tests on the effect of BP-PEG-NELL on osteoclast.

### Studies on the Bioactivity of the BP-PEG-NELL, DS S-PEG-NELL In Vivo

a. Bioactivity in healthy mouse;

b. Bioactivity in OVX rat or big animal model.

c. Characterization by micro CT, DEXA, histological staining

Those skilled in the art will know, or be able to ascertain, using no more than routine experimentation, many equivalents to the specific embodiments of the invention described herein. These and all other equivalents are intended to be encompassed by the following claim.

1. A bone-targeting therapeutic conjugate, comprising a formula of TG-M-D (I) or M-D-TG (II), wherein:
TG is a bone targeting molecule,
D is a therapeutic drug for a bone related condition, and
M is an optional chemical group that modifies the therapeutic drug.

2. The conjugate of claim 1, wherein the bone targeting molecule is a bisphosphate or DSS peptide.

3. The conjugate of claim 1, wherein the bone targeting molecule is a bisphosphonate comprising a chemical formula:
(OX)₂(O)P—(CR₁R₂)ₙ—P(O)(OM)₂, wherein:
each X is independently H, or a cation,
each R₁ is independently a hydroxyl, amino, thiol, amide, or carboxyl group,
each R₂ is a C1-C20 group comprising optional hetero atom(s), and n is an integer ranging from 1-10.

4. The conjugate of claim 3, wherein the bisphosphonate is one of:

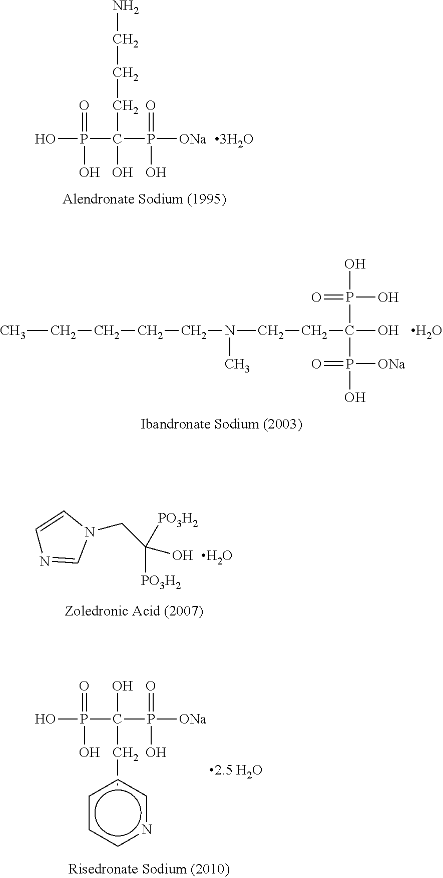

5. The conjugate of claim 1, wherein the therapeutic drug is a small molecule drug.

6. The conjugate of claim 1, wherein the therapeutic drug is a proteineous drug.

7. The conjugate of claim 1, wherein the proteineous drug is NELL-1 protein or a BMP protein.

8. The conjugate of claim 1, comprising the chemical group, wherein the chemical group comprises an alkyl group, an aryl group, an acyl group, a leaving group, a polymer, a peptide, or a combination thereof.

9. The conjugate of claim 1, comprising the chemical group, wherein the chemical group is poly(ethylene glycol) (PEG), poly(ethylene oxide) (PEO), poly(propylene glycol) (PPG), poly(propylene oxide) (PPO), heparin sulfate, glycopolymers, zwitterionic polymers, hyperbranced polymers, polymers containing unnatural amino acids, linkers to lysine or cysteine, or peptide sequences that modify drug or conjugate interactions with ECM, target cells, immune cells, and hepatocytes.

10. (canceled)

11. The conjugate of claim 1, comprising the chemical group, wherein the chemical group comprises a responsive linker that degrades on demand to external stimuli or provides a linking reaction that is azide-alkyne, azide-BMCO, or Tetrazine-TCO type reactions.

12. (canceled)

13. The conjugate of claim 1, comprising the chemical group, wherein the chemical group comprises a linker to lysine or cysteine or a natural enzyme.

14. (canceled)

15. A composition, comprising a bone-targeting therapeutic conjugate that comprises a formula of TG-M-D (I) or M-D-TG (II), wherein:
TG is a bone targeting molecule,
D is a therapeutic drug for a bone related condition, and
M is an optional chemical group that modifies the therapeutic drug.

16. (canceled)

17. The composition of claim 15, further comprises a pharmaceutically acceptable carrier.

18. The composition of claim 15, which is a formulation for systemic or local delivery.

19-23. (canceled)

24. A method of treating or ameliorating a condition in a subject in need thereof, comprising administering to the subject a bone-targeting therapeutic conjugate of a formula of TG-M-D (I) or M-D-TG (II),
wherein:
TG is a bone targeting molecule,
D is a therapeutic drug for a bone related condition, and
M is an optional chemical group that modifies the therapeutic drug.

25. (canceled)

26. The method of claim 24, wherein the conjugate is included in a composition that comprises the conjugate and a pharmaceutically acceptable carrier.

27. The method of claim 24, wherein the bone condition is osteoporosis.

28. (canceled)

29. The method of claim 24, wherein the bone condition is bone fracture or intervertebral disc disease or injury.

30. The method of claim 24, wherein administering comprises local or systemic administration.

31. The method of claim 24, wherein the subject is a human being.
